(19) United States
(12) Patent Application Publication
(10) Pub. No.: US 2022/0119773 A1
(43) Pub. Date: Apr. 21, 2022

(54) METHOD FOR IMPROVING ANGIOGENIC POTENTIAL OF A MESENCHYMAL STEM CELL

(71) Applicant: CYNATA THERAPEUTICS LIMITED, CREMORNE, VICTORIA (AU)

(72) Inventors: Kristopher KILIAN, SYDNEY (AU); Sara ROMANAZZO, SYDNEY (AU)

(73) Assignee: CYNATA THERAPEUTICS LIMITED, CREMORNE, VICTORIA (AU)

(21) Appl. No.: 17/433,579

(22) PCT Filed: Feb. 21, 2020
(86) PCT No.: PCT/AU2020/050151

§ 371 Date: Aug. 24, 2021

(30) Foreign Application Priority Data
Feb. 28, 2019 (AU) 2019900659

(51) Int. Cl. C12N 5/071; A01N 1/02; C12N 5/0775; A61K 35/44

(52) U.S. Cl. CPC C12N 5/0691; A01N 1/0284; C12N 5/0663; A61K 35/44; C12N 2506/1353; C12N 2533/52; C12N 2533/54; C12N 2539/00; C12N 2533/30

(57) ABSTRACT

The invention relates to a method for improving angiogenic potential of a mesenchymal stem cell (MSC), the method comprising culturing the MSC on a substrate having stiffness of about 1 kPa to 100 kPa and coated with a matrix protein, wherein the MSC has improved angiogenic potential when compared with a MSC cultured under identical conditions except not cultured on a substrate having stiffness of about 1 kPa to 100 kPa and not coated with a matrix protein. The invention also relates to a MSC having angiogenic potential when improved by the method, and to therapeutic use of the improved MSC for treating coronary artery disease (CAD) or peripheral artery disease (PAD) in a subject having CAD or PAD.

### FIELD

The invention relates to use of mesenchymal stem cells (MSCs) for treating coronary artery disease (CAD) and peripheral artery disease (PAD) through the trophic and immunomodulatory secretory nature of MSCs. The invention also relates to development of methods for cell engineering where substrate coatings direct pro-angiogenic secretion from MSCs.

### BACKGROUND

Coronary artery disease (CAD) and Peripheral artery disease (PAD) are the most common type of heart disease and cause most heart attacks. For example, CAD is the leading cause of death in Australia, killing one Australian every 27 minutes.

Existing angiogenesis therapies, such as direct delivery of cytokines to the site of injury, often suffer from undesirable side effects. Moreover, patients with severe nonrevascularizable CAD remain with the only option of heart transplantation, which is limited by the shortage of suitable donors.

Stem cell-based therapy emerged as a possible alternative treatment, however, limitations are related to the ability of these cells to get incorporated into the host. Targeted genetic and cell-based therapies have been explored for treatment of CAD by stimulating increased microvascular density (angiogenesis) and subsequent large vessel remodelling (arteriogenesis).

However, trials using MSCs to improve function after cardiovascular injury have had modest success due to high levels of cell death and heterogeneity in cellular response to the microenvironment. Although MSCs have demonstrated significant promise in regenerative medicine, prolonged culture (expansion) on tissue culture polystyrene hinders the secretory activity, and there has been considerable variability in clinical trials.

Thus, there is a need to improve MSC survival and MSC homogeneity.

It is to be understood that if any prior art publication is referred to herein, such reference does not constitute an admission that the publication forms a part of the common general knowledge in the art in Australia or any other country.

### SUMMARY

This disclosure relates to use of protein-conjugated hydrogel matrices as cell culture substrates to normalise the MSC secretory profile from MSCs to be pro-angiogenic (“priming”). In so doing, the disclosure relates to improved cell culture matrices that improve therapeutic efficacy of MSCs for treating CAD and PAD.

The present disclosure identifies matrix conditions that maximise secretion of pro-angiogenic factors from MSCs, as determined through model assays involving endothelial cell tubulogenesis. Surprisingly, MSCs cultured on the disclosed matrices may be cryopreserved under liquid nitrogen, and following thawing, maintain the primed pro-angiogenic phenotype.

Directing a desired cell activity through substrate properties alone has many advantages over methods using hypoxia or growth factor treatment, including simplicity of manufacture and minimal modifications to the cell source.

MSCs produced according to this disclosure have a pro-angiogenic secretome and are useful in treating CAD and PAD.

A first aspect provides a method for improving angiogenic potential of a mesenchymal stem cell (MSC), the method comprising culturing the MSC on a substrate having stiffness of about 1 kPa to 100 kPa and coated with a matrix protein, wherein the MSC has improved angiogenic potential when compared with a MSC cultured under identical conditions except not cultured on a substrate having stiffness of about 1 kPa to 100 kPa and not coated with a matrix protein.

Also disclosed is a method for preparing a mesenchymal stem cell (MSC) having improved angiogenic potential, the method comprising culturing the MSC on a substrate having stiffness of about 1 kPa to 100 kPa and coated with a matrix protein, wherein the MSC has improved angiogenic potential when compared with a MSC cultured under identical conditions except not cultured on a substrate having stiffness of about 1 kPa to 100 kPa and not coated with a matrix protein.

The method may be in vitro.

In one embodiment, the stiffness is about, 1 kPa, 10 kPa, or 40 kPa.

In one embodiment, the matrix protein is a collagen, fibronectin, or laminin.

In one embodiment, the substrate has stiffness of about 10 kPa and is coated with fibronectin.

In one embodiment, the substrate has stiffness of about 1 kPa or 10 kPa and is coated with fibronectin and collagen.

In one embodiment, the substrate is coated with a matrix protein at about 25 μg/mL.

In one embodiment, the substrate comprises polyacrylamide.

In one embodiment, the MSC is produced according to WO2017/156580.

In one embodiment, the method further comprises cryopreserving the MSC after culturing the MSC on the substrate.

In one embodiment, the method further comprises thawing the cryopreserved MSC, wherein improved angiogenic potential persists after cryopreservation and thawing.

In one embodiment, improved angiogenic potential is measured using a tubulogenesis assay.

A second aspect provides a mesenchymal stem cell (MSC) having angiogenic potential when improved by the method of the first aspect.

A third aspect provides a composition comprising a mesenchymal stem cell (MSC) when prepared by a method comprising culturing the MSC on a substrate having stiffness of about 1 kPa to 100 kPa and coated with a matrix protein, wherein the MSC has improved angiogenic potential when compared with a MSC cultured under identical conditions except not cultured on a substrate having stiffness of about 1 kPa to 100 kPa and not coated with a matrix protein.

In one embodiment, the composition of the third aspect is a pharmaceutical composition comprising a pharmaceutically acceptable carrier, diluent and/or excipient.

A fourth aspect provides a container comprising the MSC of the second aspect or the composition of the third aspect.

A fifth aspect provides a kit comprising the MSC the second aspect or the composition of the third aspect, or the container of the fourth aspect.

A sixth aspect provides a method for treating coronary artery disease (CAD) or peripheral artery disease (PAD), the method comprising administering to a subject having CAD or PAD the MSC of the second aspect.

Additionally or alternatively, the sixth aspect provides use of the MSC of the second aspect in the manufacture of a medicament for treating coronary artery disease (CAD) or peripheral artery disease (PAD) in a subject having CAD or PAD.

Additionally or alternatively, the sixth aspect provides the MSC of the second aspect for use in a method for treating coronary artery disease (CAD) or peripheral artery disease (PAD) in a subject having CAD or PAD.

### BRIEF DESCRIPTION OF FIGURES

FIG. 1 is a schematic representation of the experimental design investigating matrix biological and physical composition influence in stem cell proangiogenesis.

FIG. 2 is a schematic representation of the experimental design testing the persistence of the pro-angiogenic effects in primed MSCs after cryopreservation.

FIG. 3 is a schematic representation of the tubulogenesis assay analyses. Master segments are shown in yellow and consist in pieces of tree delimited by two junctions none exclusively implicated with one branch, called master junctions. Master junctions are junctions linking at least three master segments. Optionally, two close master junctions can be fused into a unique master junction. Master junctions are shown in red. Meshes are areas enclosed by segments or master segments. Meshes are shown in blue.

FIG. 4 are photomicrographs showing that the matrix biological and physical composition affects MSC morphology. MSCs cultured on polyacrylamide gels with different coatings, showed different cell shape and actin filament organization (in red) depending on substrate stiffness (1 kPa left, 10 kPa middle, and 40 kPa right) and on ECM proteins conjugated to each substrate (Collagen, top; Fibronectin, middle; Laminin, bottom). Nuclei were counterstained with DAPI, 4-6-diamidino-2-phenylindole.

FIG. 5 are column graphs depicting the results of the tubulogenesis assay measuring tube formation in which HMVECs were treated with conditioned media from MSCs cultured across varying stiffness hydrogels and matrix protein composition. A total length of master segments; B total length of branches; C total length; D total length of segments.

FIG. 6. (A) Polyacrylamide gel fabrication and conjugation (B) Average human microvascular endothelial cell (HMVEC) tube area after treatment with conditioned media from MSCs cultured across varying stiffness hydrogels and ligand composition. (C) Images of HMVECs under positive and negative controls. (C) (top) HMVECs cultured under media from the Fibronectin 0.5, 10 and 40 kPa conditions respectively, (bottom) substrate stiffness changes MSC cell spreading characteristics and affects their secretory profiles. * indicates p<0.05.

FIG. 7 are phase contrast photomicrographs of HMVEC culture with media from standard tissue culture plates (TCPS) coating with a combination of fibronectin and collagen I (left), 1 kPa collagen (centre) and 10 kPa fibronectin (right), and a column graph quantifying the three conditions. *p<0.05.

FIG. 8 are column graphs depicting the results of the tubulogenesis assay measuring total length of master segments in HMVECs were treated with conditioned media from MSCs cultured across varying stiffness hydrogels and matrix protein composition, before (left) and after (right) cryopreservation. Primed MSCs maintained their ability to induce tube formation after cryopreservation. Left, *p<0.05. Right, p<0.05 by one-way ANOVA.

FIG. 9 provides a schematic representation of the tubulogenesis assay after culturing MSCs on a hydrogel coated with two matrix proteins, the quantification of the tubulogenesis assay, and phase contrast photomicrographs of each condition showing tubule formation. Prior to MSC culture, the hydrogel was coated with a combination of fibronectin 12.5 μg/mL and collagen 12.5 μg/mL. The combination of two matrix proteins increased the angiogenesis potential of the MSCs after cryopreservation.

### DETAILED DESCRIPTION

“Coronary artery disease” or “CAD” refers to the narrowing of the coronary arteries reducing blood flow, hence oxygen supply, to the heart. CAD may also be referred to as “coronary heart disease” or “CHD”.

“Peripheral artery disease” or “PAD” refers to the narrowing of arteries supplying blood, hence oxygen, to the limbs.

“Atherosclerosis” encompasses both CAD and PAD, so the present disclosure is also relevant to treating atherosclerosis.

As used herein, “mesenchymal stem cell” or “MSC” refers to a particular type of stem cell that may be isolated from a wide range of tissues, including bone marrow, adipose tissue (fat), placenta and umbilical cord blood. MSCs are also known as “mesenchymal stromal cells”.

MSCs secrete bioactive molecules such as cytokines, chemokines and growth factors and are able to modulate the immune system. MSCs have been shown to facilitate regeneration and effects on the immune system without relying upon engraftment. In other words, the MSCs themselves do not necessarily become incorporated into the host—rather, they exert their effects and are then eliminated within a short period of time. However, MSCs may be engrafted.

Therapeutic MSCs can be either “autologous” or “allogeneic”. As used herein, “autologous” means a patient is treated with their own cells isolated from bone marrow or adipose tissue, for example, whereas “allogeneic” means that cells from a donor are used to treat other people. Allogeneic MSCs may be derived from a donor via an induced pluripotent stem cell or iPSC. Alternatively, allogeneic MSCs may be derived from an embryonic stem cell or ESC. Otherwise, allogeneic MSCs may also be derived from other sources, including for example donor bone marrow, adipose tissue, umbilical cord tissue Jr blood, Cr molar cells such as developing tooth bud of the mandibular third molar.

Allogeneic MSCs have not been shown to cause immune reactions in other people, so they do not require immune-matching the donor to the recipient. This has important commercial advantages.

As used herein, “pluripotent stem cell” or “PSC” refers to a cell that has the ability to reproduce itself indefinitely, and to differentiate into any other cell type. There are two main types of pluripotent stem cell: embryonic stem cells (ESCs) and induced pluripotent stem cells (iPSCs).

As used herein, “embryonic stem cell” or “ESC” refers to a cell isolated from a five to seven day-old embryo donated with consent by patients who have completed in vitro fertilisation therapy, and have surplus embryos. The use of ESCs has been hindered to some extent by ethical concerns about the extraction of cells from human embryos.

Suitable human PSCs include H1 and H9 human embryonic stem cells (hESCs). H1 and H9 hESCs are available from WiCell, Madison, Wis. 53719 USA, for example.

As used herein, “induced pluripotent steal cell” or “iPSC” refers to an ESC-like cell derived from adult cells. iPSCs have very similar characteristics to ESCs, but avoid the ethical concerns associated with ESCs, since iPSCs are not derived from embryos. Instead, iPSCs are typically derived from fully differentiated adult cells that have been “reprogrammed” back into a pluripotent state.

Suitable human iPSCs include, but are not limited to, iPSC 19-9-7T, MIRJT6i-mND1-4 and MIRCIT7i-mND2-0 derived from fibroblasts and iPSC EM119-9 derived from bone marrow mononuclear cells are available from WiCell, Madison, Wis. 53719 USA, for example. Other suitable iPSCs may be obtained from Cellular Dynamics international of Madison, Wis., USA.

According to one embodiment of the present disclosure, MSCs are formed from ^{EMH}lin⁻KDR⁺APLNR⁺PDGFRalpha⁺ primitive mesoderm cells with mesenchymoangioblast (MCA) potential, and may be produced according to WO2017/156580. WO2017/156580 is hereby incorporated by reference in its entirety.

Human MSCs produced according to WO2017/156580 and optionally assayed according to WO2018/090084 may be subject to angiogenic priming according to the present disclosure. Other MSCs known to the person skilled in the art may be subject to angiogenic priming according to the present disclosure.

Matrix proteins may comprise an extracellular matrix (ECM) protein. Matrix proteins may comprise: laminin; a collagen, for example collagen I or collagen IV; fibronectin; elastin; a proteoglycan, for example heparan sulfate, chondroitin sulfate, or keratan sulfate. A matrix protein may be mammalian. A matrix protein may be human or non-human mammalian. The person skilled in the art will be aware of these and other matrix proteins.

The substrate or hydrogel may be coated with two or more matrix proteins.

The substrate or hydrogel may be coated with the matrix protein at about or ±10% of 1, 2, 2.5, 3, 4, 5, 6, 7, 7.5, 8, 9, 10, 11, 12, 12.5, 13, 14, 15, 16, 17, 17.5, 18, 19, 20, 21, 22, 22.5, 23, 24, 25, 30, 35, 40, 45, 50, 55, 60, 65, 70, 75, 80, 85, 90, 95 or 100 μg/mL. In one embodiment, collagen is coated on the substrate or hydrogel at 12.5 μg/mL. In one embodiment, fibronectin is coated on the substrate or hydrogel at 12.5 μg/mL.

Substrate or hydrogel formulations spanning about or ±10% 1 kPa to 100 kPa stiffness may be used to prime the MSCs in culture. For example, hydrogel formulations of about or ±10% of 1, 2, 3, 4, 5, 6, 7, 8, 9, 10, 11, 12, 13, 14, 15, 16, 17, 18, 19 20, 21, 22, 23, 24, 25, 30, 35, 40, 45, 50, 55, 60, 65, 70, 75, 80, 85, 90, 95 or 100 kPa. 1 kPa to 100 kPa stiffness spans the range of normal and pathological heart tissue stiffness.

The substrate or hydrogel may comprise polyvinyl alcohol, sodium polyacrylate, acrylate polymers and copolymers with an abundance of hydrophilic groups, or a naturally occurring hydrogel such as agarose, methylcellulose, hyaluronan, or elastin-like polypeptides. In one embodiment, the hydrogel comprises polyacrylamide.

In one embodiment, the substrate or hydrogel has stiffness of about or ±10% 1 kPa and is coated with collagen. In another embodiment, the hydrogel has stiffness of about or ±10% 10 kPa and is coated with fibronectin. In another embodiment, the hydrogel has stiffness of about or ±10% 1 kPa to 10 kPa, 1 kPa or 10 kPa and is coated with fibronectin and collagen.

The MSCs may be cultured on the substrate coated with the matrix protein for around or ±10% 1, 2, 3, 4, 5, 6, 7, 8, 9, 10, 11, 12, 13, or 14 days, for example. In one embodiment, the MSCs are cultured on the substrate coated with the matrix protein for around or ±10% 2 days.

“Angiogenesis” refers to the formation of new blood vessels from the endothelial cells (ECs) of pre-existing veins, arteries, and capillaries.

It follows that “angiogenic potential” refers to the potential or capacity of an MSC to promote angiogenesis.

As used herein, “improved” angiogenic potential refers to an increased potential or capacity of a MSC, e.g. a test MSC produced according to the disclosure, to promote angiogenesis when compared with a MSC cultured under identical conditions except not cultured on a substrate having stiffness of about 1 kPa to 100 kPa and not coated with a matrix protein, e.g. a reference or control MSC, wherein angiogenic potential of a test MSC and a reference MSC is measured objectively using an angiogenesis assay. In other words, a MSC of the disclosure has improved angiogenic potential when compared to its reference or control MSC. The terms “reference” and “control” will be understood by the person skilled in the art.

Angiogenesis assays may be used to evaluate angiogenic potential. An angiogenesis assay may be in vitro or in vivo. In general, in vitro assays monitor specific stages in the angiogenesis process. An angiogenesis assay may evaluate: proliferation (e.g. involving cell counting, colorimetry, or by DNA synthesis); migration (e.g. involving wound healing, human dermal microvascular endothelial cell (HDMEC) sprouting, matrix degradation, a Boyden chamber, phagokinetic track); tube formation (e.g. involving MATRIGEL, co-culture); a thoracic aorta ring; a retina model; a chick chorioallantoic membrane; zebrafish; corneal angiogenesis; xenograft; or a MATRIGEL plug. Angiogensis assays are available commercially.

As will be understood by the person skilled in the art, the tubulogenesis assay employed herein is accepted in the art as an in vitro assay that is indicative of angiogenesis. Tubulogenesis in the assay may be quantified at around or ±10% 1, 2, 4, 8, or 16 h, for example.

The terms “substrate”, “matrix” and “hydrogel”, for example, are used interchangeably herein and are not to be considered limited unless the contrary is clearly intended.

The terms “stiffness” (or “stiff”) and “rigidity” or (“rigid”), for example, are used interchangeably herein and are not to be considered limited.

An MSC of the disclosure or a composition comprising an MSC of the disclosure may be administered by parenteral routes (e.g., intravenous, intraarterial, subcutaneous, intraperitoneal, intramuscular, or transdermal). In one embodiment, the MSC or pharmaceutical composition is administered intravenously or intraarterially.

An MSC of the disclosure or a pharmaceutical composition comprising an MSC of the disclosure may be administered to a subject alone or in combination with a pharmaceutically acceptable carrier, diluent and/or excipient in single or multiple doses.

Pharmaceutical compositions of the present disclosure can be prepared by methods well known in the art (e.g., Remington: The Science and Practice of Pharmacy, 21st ed. (2005), A. Gennaro et al., Lippincott Williams & Wilkins) and comprise an MSC as disclosed herein, and one or more pharmaceutically acceptable carriers, diluents, and/or excipients.

Also provided is an article of manufacture and/or a kit, comprising a container comprising an MSC of the disclosure or a pharmaceutical composition comprising an MSC of the disclosure. The container may be a bottle, vial or syringe comprising MSC of the disclosure or a pharmaceutical composition comprising an MSC of the disclosure, optionally in unit dosage form. For example, MSC of the disclosure or a pharmaceutical composition comprising an MSC of the disclosure may be injectable in a disposable container, optionally a syringe. The article of manufacture and/or kit may further comprise printed instructions and/or a label or the like, indicating treatment of a subject according to the method disclosed herein.

A “unit dosage form” can be created to facilitate administration and dosage uniformity and refers to physically discrete units suited as single dosages for the subject to be treated, containing a therapeutically effective quantity of an MSC of the disclosure or a pharmaceutical composition comprising an MSC of the disclosure in association with the required pharmaceutical excipient, carrier and/or diluent. In one embodiment, the unit dosage form is a sealed container and is sterile.

The term “therapeutically effective amount” refers to an amount of MSC of the disclosure or a pharmaceutical composition comprising an MSC of the disclosure effective to treat CAD or PAD in a subject.

The terms “treat”, “treating” or “treatment” refer to both therapeutic treatment and prophylactic or preventative measures, wherein the aim is to prevent, reduce, or ameliorate CAD or PAD in a subject or slow down (lessen) progression of CAD or PAD in a subject. Subjects in need of treatment include those already with CAD or PAD as well as those in which CAD or PAD is to be prevented or ameliorated.

The terms “preventing”, “prevention”, “preventative” or “prophylactic” refers to keeping from occurring, or to hinder, defend from, or protect from the occurrence of CAD or PAD. A subject in need of prevention may be prone to develop CAD or PAD.

The term “ameliorate” or “amelioration” refers to a decrease, reduction or elimination of CAD or PAD.

As used herein, the term “subject” may refer to a mammal. The mammal may be a primate, particularly a human, or may be a domestic, zoo, or companion animal. Although it is particularly contemplated that the MSCs, compositions and method disclosed herein are suitable for medical treatment of humans, it is also applicable to veterinary treatment, including treatment of domestic animals such as horses, cattle and sheep, companion animals such as dogs and cats, or zoo animals such as felids, canids, bovids and ungulates.

Unless defined otherwise in this specification, technical and scientific terms used herein have the same meaning as commonly understood by the person skilled in the art to which this invention belongs and by reference to published texts.

In the claims which follow and in the description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

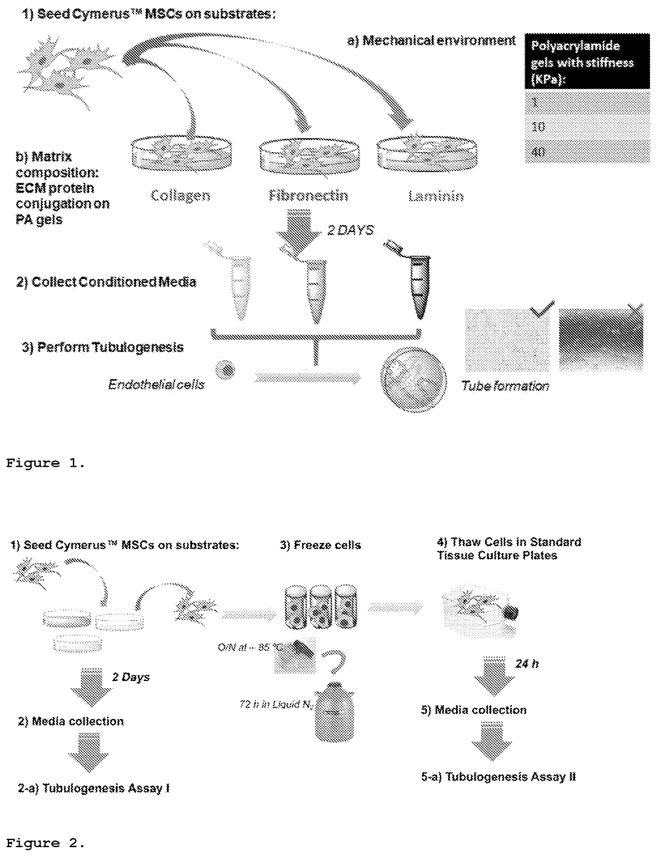

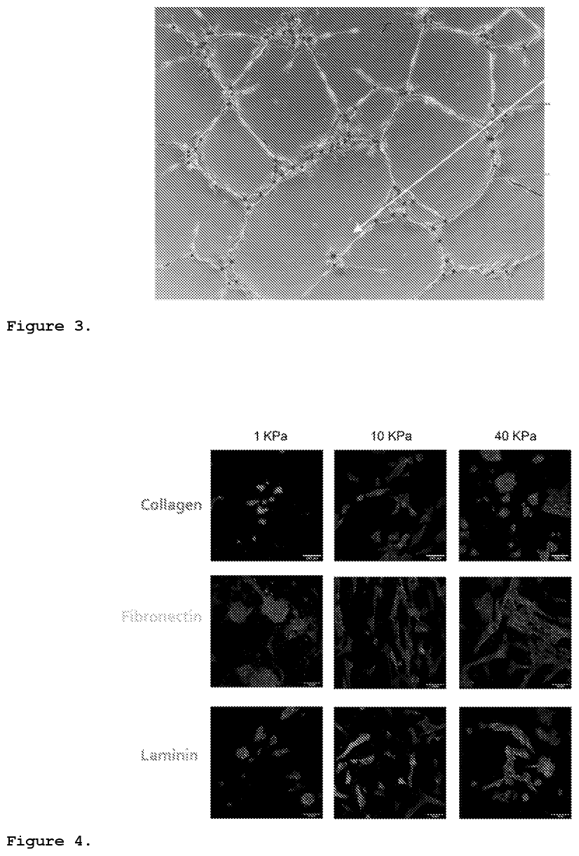

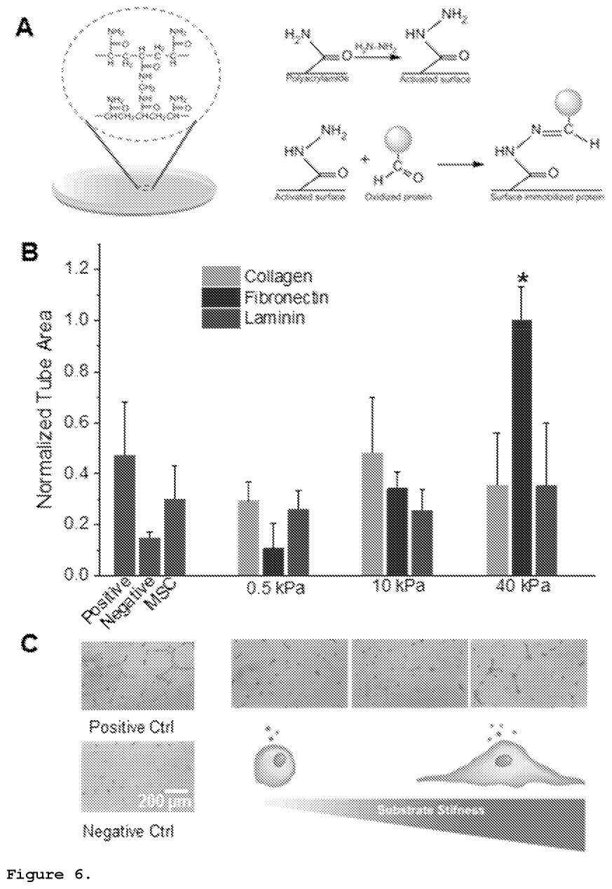

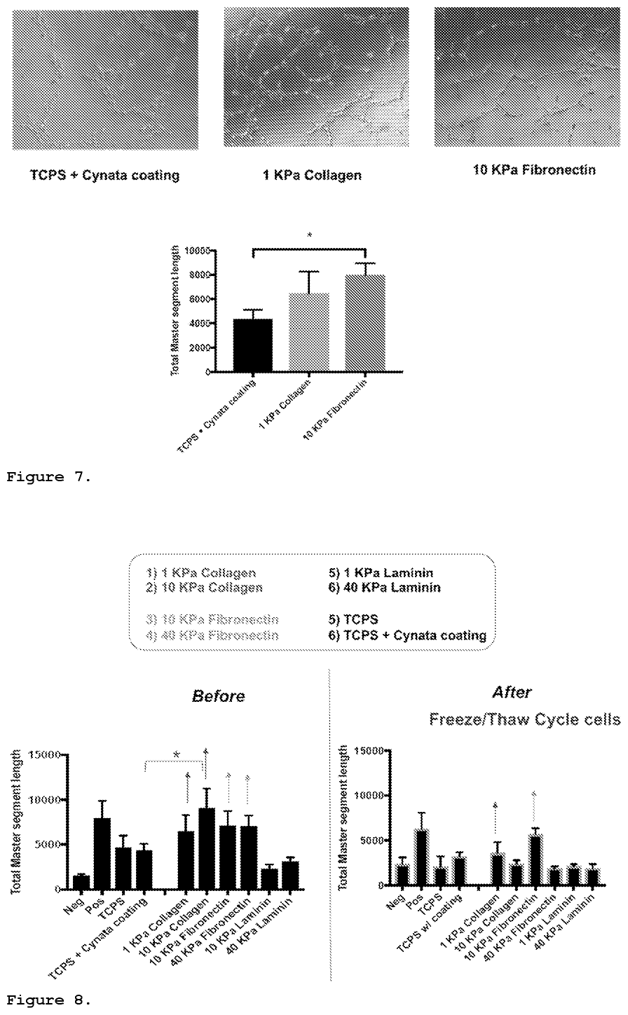

The experimental design is depicted in FIG. 1 and FIG. 2. Results of the experiments are depicted in FIGS. 3 to 9.

The figures show that the Matrix biological and physical composition affected MSC morphology. MSCs appeared different, in terms of cell shape and actin filament organization, depending on gel stiffness and on the proteins conjugated to each substrate (FIG. 4). Cells showed a rounded morphology in all conditions and more pronounced cell aggregation in 1 kPa fibronectin group (FIG. 4, middle left). On higher stiffness gels, MSCs were spread. In particular, cells seeded on 10 kPa fibronectin substrates were able to align to each other (FIG. 4, middle line, centre). Cells cultured on collagen coated surfaces maintained cell aggregation on higher rigidity substrates also.

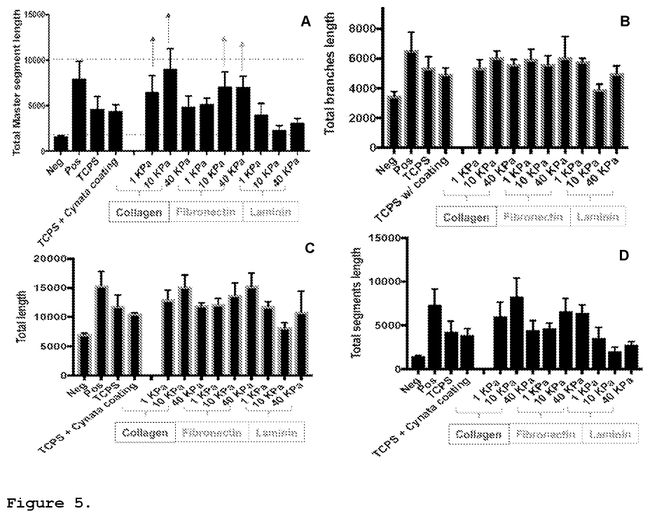

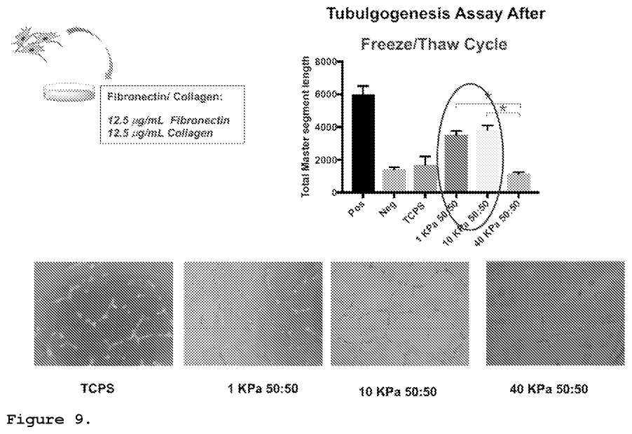

The figures also show that tube formation is stimulated by a combination of specific substrate stiffness and matrix protein. After 2 days culture on the hydrogels, cell culture media was collected from each condition and used to perform a tubulogenesis assay. Tube formation was assessed after 8 h. Results showed that all collagen and fibronectin coated surfaces were able to induce tube formation better than the normal tissue culture plates (TCPS) and the TCPS coated with the combination of fibronectin and collagen I. Moreover, conditioned media from 10 kPa collagen showed higher tube formation than positive control (FIG. 5). When fibronectin and collagen were both coated on varying stiffness gels, 1 kPa and 10 kPa showed best tubulogenesis (FIG. 9).

### EXAMPLES

### Example 1

Human MSCs produced according to WO2017/156580 and optionally assayed according to WO2018/090084 were used.

For hydrogel conjugation, polydimethylsiloxane (PDMS) stamps were fabricated using photo-lithography for printing of oxidized protein onto polyacrylamide. Hydrogel formulations spanning 1-40 kPa were investigated; hydrogel mechanical properties were verified through nanoindentation. Matrix proteins laminin, collagen I, and fibronectin were oxidized and patterned on the substrates alone and in combinations. Protein surface density was verified using iodination.

Conditioned media from the MSCs was collected after 2 days. Angiogenic activity was probed using an in vitro tubulogenesis assay, where conditioned media was added to growth-factor depleted matrigel containing human microvascular endothelial cells (hMVECs). Images of tube formation were collected at 8 hours and quantified using ImageJ (NIH).

Conditions that primed a pro-angiogenic state were investigated for persistence of the activated state before and after cryopreservation.

### Example 2

MSC-conditioned media that promote tubulogenesis will be profiled for a panel of pro-angiogenic cytokines using a commercially available cytokine array.

MSCs will be encapsulated within a poly(ethylene glycol) diacrylate (PEGDA) hydrogel crosslinked with matrix metalloprotease (MMP) degradable peptides. Proteins identified in the screen that promote an angiogenic secretome with be acrylated for incorporation within the material. Mechanical properties will be tuned through PEGDA molecular weight and evaluated with nanoindentation. Antibody arrays and in vitro tubulogenesis of HMVECs will be used to evaluate secretion from the encapsulated MSCs.

### Example 3—Protocol for Differentiating a Human PSC into a MSC

TABLE 1
Reagents

| Description | Vendor/Cat # or Ref # |
|---|---|
| DMEM/F12 Base Medium | Invitrogen/A1516901 |
| E8 supplement | Invitrogen/A1517101 |
| vitronectin | Life Technologies/A14700 |
| collagen IV | Sigma/C5533 |
| H-1152 ROCK Inhibitor | EMD Millipore/555550 |
| Y27632 dihydrochloride ROCK | Tocris/1254 |
| Inhibitor | |
| FGF2 | Waisman Biomanufacturing/ |
| | WC-FGF2-FP |
| human endothelial-SFM | Life Technologies/11111-044 |
| stemline II hematopoietic stem cell | Sigma/S0192 |
| expansion medium | |
| GLUTAMAX | Invitrogen/35050-061 |
| insulin | Sigma/I9278 |
| lithium chloride (LiCl) | Sigma/L4408 |
| collagen I solution | Sigma/C2249 |
| fibronectin | Life Technologies/33016-015 |
| DMEM/F12 | Invitrogen/11330032 |
| recombinant human BMP4 | Peprotech/120-05ET |
| activin A | Peprotech/120-14E |
| Iscove's modified Dulbecco's medium | Invitrogen/12200036 |
| (IMDM) | |
| Ham's F12 nutrient mix | Invitrogen/21700075 |
| sodium bicarbonate | Sigma/S5761 |
| L-ascorbic acid 2-phosphate Mg²⁺ | Sigma/A8960 |
| 1-thioglycerol | Sigma/M6145 |
| sodium selenite | Sigma/S5261 |
| non essential amino acids | HyClone/SH30853.01 |
| chemically defined lipid | Invitrogen/11905031 |
| concentrate | |
| embryo transfer grade water | Sigma/W1503 |
| polyvinyl alcohol (PVA) | MP Bio/151-941-83 |
| holo-transferrin | Sigma/T0665 |
| ES-CULT M3120 | Stem Cell Technologies/03120 |
| STEMSPAN serum-free expansion | Stem Cell Technologies/09650 |
| medium (SFEM) | |
| L-ascorbic acid | Sigma/A4544 |
| PDGF-BB | Peprotech/110-14B |

The reagents listed in Table 1 are known to the person skilled in the art and have accepted compositions, for example IMDM and Ham's F12. GLUTAMAX comprises L-alanyl-L-glutamine dipeptide, usually supplied at 200 mM in 0.85% NaCl. GLUTAMAX releases L-glutamine upon cleavage of the dipeptide bond by the cells being cultured. Chemically defined lipid concentrate comprises arachidonic acid 2 mg/L, cholesterol 220 mg/L, DL-alpha-tocopherol acetate 70 mg/L, linoleic acid 10 mg/L, linolenic acid 10 mg/L, myristic acid 10 mg/L, oleic acid 10 mg/L, palmitic acid 10 mg/L, palmitoleic acid 10 mg/L, pluronic F-68 90 g/L, stearic acid 10 mg/L, TWEEN 80® 2.2 g/L, and ethyl alcohol. H-1152 and Y27632 are highly potent, cell-permeable, selective ROCK (Rho-associated coiled coil forming protein serine/threonine kinase) inhibitors.

TABLE 2
IF6S medium (10X concentration)

| | | | Final | |
|---|---|---|---|---|
| 10X IF6S | Quantity | | Concentration | |
| IMDM | 1 package, | | 5X | |
| | powder for 1 L | | | |
| Ham's F12 nutrient mix | 1 package, | | 5X | |
| | powder for 1 L | | | |
| sodium bicarbonate | 4.2 | g | 21 | mg/mL |
| L-ascorbic acid 2-phosphate Mg²⁺ | 128 | mg | 640 | μg/mL |
| 1-thioglycerol | 80 | μL | 4.6 | mM |
| sodium selenite (0.7 mg/mL solution) | 24 | μL | 84 | ng/mL |

| | | | |
|---|---|---|---|
| GLUTAMAX | 20 | mL | 10X |
| non essential amino acids | 20 | mL | 10X |
| chemically defined lipid concentrate | 4 | mL | 10X |
| embryo transfer grade water | To 200 | mL | NA |

TABLE 3
IF9S medium (1X concentration; based on IF6S)

| | | | Final | |
|---|---|---|---|---|
| IF9S | Quantity | | Concentration | |
| IF6S | 5 | mL | 1X | |
| polyvinyl alcohol (PVA; | 25 | mL | 10 | mg/mL |
| 20 mg/mL solution) | | | | |
| holo-transferrin (10.6 | 50 | μL | 10.6 | μg/mL |
| mg/mL solution) | | | | |
| insulin | 100 | μL | 20 | μg/mL |

| | | | |
|---|---|---|---|
| embryo transfer grade water | To 50 | mL | NA |

TABLE 4
Differentiation medium (1X concentration;
based on IF9S)

| | | | Final | |
|---|---|---|---|---|
| Differentiation Medium | Quantity | | Concentration | |
| IF9S | 36 | mL | 1X | |
| FGF2 | 1.8 | μg | 50 | ng/mL |
| LiCl (2M solution) | 36 | μL | 2 | mM |
| BMP4 (100 μg/mL solution) | 18 | μL | 50 | ng/mL |
| Activin A (10 mg/mL solution) | 5.4 | μL | 1.5 | ng/mL |

TABLE 5
Mesenchymal colony forming medium (1X concentration)

| | | | Final | |
|---|---|---|---|---|
| M-CFM | Quantity | | Concentration | |
| ES-CULT M3120 | 40 | mL | 40% | |
| STEMSPAN SFEM | 30 | mL | 30% | |
| human endothelial-SFM | 30 | mL | 30% | |
| GLUTAMAX | 1 | mL | 1X | |
| L-ascorbic acid (250 mM solution) | 100 | μL | 250 | μM |
| LiCl (2M solution) | 50 | μL | 1 | mM |
| 1-thioglycerol (100 mM solution) | 100 | μL | 100 | μM |
| FGF2 | 600 | ng | 20 | ng/mL |

TABLE 6
Mesenchymal serum-free expansion medium
(1X concentration)

| | | | Final | |
|---|---|---|---|---|
| M-SFEM | Quantity | | Concentration | |
| human endothelial-SFM | 5 | L | 50% | |
| STEMLINE II HSFM | 5 | L | 50% | |
| GLUTAMAX | 100 | mL | 1X | |
| 1-thioglycerol | 87 | μL | 100 | μM |
| FGF2 | 100 | μg | 10 | ng/mL |

### Protocol

1. Thawed iPSCs in E8 Complete Medium (DMEM/F12 Base Medium+E8 Supplement)+1 μM H1152 on Vitronectin coated (0.5 μg/cm²) plastic ware. Incubated plated iPSCs at 37° C., 5% CO₂, 20% O₂ (normoxic).
2. Expanded iPSCs three passages in E8 Complete Medium (without ROCK inhibitor) on Vitronectin coated (0.5 μg/cm²) plastic ware and incubated at 37° C., 5% CO₂, 20% O₂ (normoxic) prior to initiating differentiation process.
3. Harvested and seeded iPSCs as single cells/small colonies at 5×10³ cells/cm² on Collagen IV coated (0.5 μg/cm²) plastic ware in E8 Complete Medium+10 μM Y27632 and incubated at 37° C., 5% CO₂, 20% O₂ (normoxic) for 24 h.
4. Replaced E8 Complete Medium+10 μM Y27632 with Differentiation Medium and incubated at 37° C., 5% CO₂, 5% O₂ (hypoxic) for 48 h.
5. Harvested colony forming cells from Differentiation Medium adherent culture as a single cell suspension, transferred to M-CFM suspension culture and incubated at 37° C., 5% CO₂, 20% O₂ (normoxic) for 12 days.
6. Harvested and seeded colonies (Passage 0) on Fibronectin/Collagen I coated (0.67 μg/cm² Fibronectin, 1.2 μg/cm² Collagen I) plastic ware in M-SFEM and incubated at 37° C., 5% CO₂, 20% O₂ (normoxic) for 3 days.
7. Harvested colonies and seeded as single cells (Passage 1) at 1.3×10⁴ cells/cm² on Fibronectin/Collagen 1 coated plastic ware in M-SFEM and incubated at 37° C., 5% CO₂, 20% O₂ (normoxic) for 3 days.
8. Harvested and seeded as single cells (Passage 2) at 1.3×10⁴ cells/cm² on Fibronectin/Collagen 1 coated plastic ware in M-SFEM and incubated at 37° C., 5% CO₂, 20% O₂ (normoxic) for 3 days.
9. Harvested and seeded as single cells (Passage 3) at 1.3×10⁴ cells/cm² on Fibronectin/Collagen 1 coated plastic ware in M-SFEM and incubated at 37° C., 5% CO₂, 20% O₂ (normoxic) for 3 days.
10. Harvested and seeded as single cells (Passage 4) at 1.3×10⁴ cells/cm² on Fibronectin/Collagen 1 coated plastic ware in M-SFEM and incubated at 37° C., 5% CO₂, 20% O₂ (normoxic) for 3 days.
11. Harvested and seeded as single cells (Passage 5) at 1.3×10⁴ cells/cm² on Fibronectin/Collagen 1 coated plastic ware in M-SFEM and incubated at 37° C., 5% CO₂, 20% O₂ (normoxic) for 3 days.
12. Harvested as single cells and froze final product.

1. A method for improving angiogenic potential of a mesenchymal stem cell (MSC), the method comprising culturing the MSC on a substrate having stiffness of about 1 kPa to 100 kPa and coated with a matrix protein, wherein the MSC has improved angiogenic potential when compared with a MSC cultured under identical conditions except not cultured on a substrate having stiffness of about 1 kPa to 100 kPa and not coated with a matrix protein.

2. The method of claim 1, wherein the stiffness is about, 1 kPa, 10 kPa, or 40 kPa.

3. The method of claim 1, wherein the matrix protein is a collagen, fibronectin, or laminin.

4. The method of claim 1, wherein the substrate has stiffness of about 10 kPa and is coated with fibronectin.

5. The method of claim 1, wherein the substrate has stiffness of about 1 kPa or 10 kPa and is coated with fibronectin and collagen.

6. The method of claim 1, wherein the substrate is coated with a matrix protein at about 25 μg/mL.

7. The method of claim 1, wherein the substrate comprises polyacrylamide.

8. The method of claim 1, wherein the MSC is produced according to WO2017/156580.

9. The method of claim 1, further comprising cryopreserving the MSC after culturing the MSC on the substrate.

10. The method of claim 9, further comprising thawing the cryopreserved MSC, wherein improved angiogenic potential persists after cryopreservation and thawing.

11. The method of claim 1, wherein improved angiogenic potential is measured using a tubulogenesis assay.

12. A mesenchymal stem cell (MSC) having angiogenic potential when improved by the method of claim 1.

13. A composition comprising a mesenchymal stem cell (MSC) when prepared by a method comprising culturing the MSC on a substrate having stiffness of about 1 kPa to 100 kPa and coated with a matrix protein, wherein the MSC has improved angiogenic potential when compared with a MSC cultured under identical conditions except not cultured on a substrate having stiffness of about 1 kPa to 100 kPa and not coated with a matrix protein.

14. The composition of claim 13, wherein the composition is a pharmaceutical composition comprising a pharmaceutically acceptable carrier, diluent and/or excipient.

15. A container comprising the MSC of claim 12.

16. A kit comprising the MSC of claim 12.

17. A method for treating coronary artery disease (CAD) or peripheral artery disease (PAD), the method comprising administering to a subject having CAD or PAD.

18. (canceled)

19. (canceled)
